(19) United States
(12) Patent Application Publication
(10) Pub. No.: US 2022/0339038 A1
(43) Pub. Date: Oct. 27, 2022

(54) SMART PROTECTIVE FACE MASK

(71) Applicant: Roi Greene, Philadelphia, PA (US)

(72) Inventor: Roi Greene, Philadelphia, PA (US)

(21) Appl. No.: 17/684,547

(22) Filed: Mar. 2, 2022

Related U.S. Application Data
(60) Provisional application No. 63/178,651, filed on Apr. 23, 2021.

(51) Int. Cl. A61F 9/02; A41D 13/11; A62B 18/00; A62B 23/02; H04B 1/38; H04R 1/02; H04R 3/00

(52) U.S. Cl. CPC A61F 9/029; A41D 13/11; A62B 18/003; A62B 23/02; H04B 1/38; H04R 1/028; H04R 3/00; H04R 2420/09

(57) ABSTRACT

A protective face mask that includes a power source, ventilation mechanisms, filtering mechanisms, audio and video mechanisms, headphones, a lighting mechanism, a night vision mechanism, a visual zooming mechanism, protective eyewear, a head canopy, a thermometer, a DNA testing mechanism, virus detection mechanisms, radio communication technology, and fiber optics technology.

### CROSS-REFERENCE TO RELATED APPLICATION

There present application is related to and claims priority from prior provisional application Ser. No. 63/178,651, filed Apr. 23, 2021 which is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

### 1. Field of the Invention

The present invention relates generally to the field of protective and filtering face masks.

### 2. Description of the Related Art

The prior art masks do not adequately allow people to hear each other when wearing protective face masks. Prior art protective face masks are usually difficult to breathe through. Furthermore, prior art protective face masks do not provide additional features such a power source, audio and video mechanisms, headphones, a lighting mechanism, a night vision mechanism, a visual zooming mechanism, protective eyewear, a head canopy, a thermometer, a DNA testing mechanism, virus detection mechanisms, radio communication technology, and fiber optics technology. Thus, a need exists for a protective face masks to avoid the above-mentioned problems.

### BRIEF SUMMARY OF THE INVENTION

In view of the foregoing disadvantages inherent in the known protective face masks and information readily available art, the present invention provides a novel smart protective face mask. The general purpose of the present invention, which will be described subsequently in greater detail, is to provide a smart protective face mask that includes a power source, ventilation mechanisms, filtering mechanisms, audio and video mechanisms, headphones, a lighting mechanism, a night vision mechanism, a visual zooming mechanism, protective eyewear, a head canopy, a thermometer, a DNA testing mechanism, virus detection mechanisms, radio communication technology, and fiber optics technology.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

### BRIEF DESCRIPTION OF THE DRAWINGS

The drawings that accompanies the written portion of this specification illustrates an embodiment of the present invention, A Smart Protective Face Mask, constructed and operative according to the teachings of the present invention.

FIG. 1 shows a front perspective view of the smart protective face mask according to the preferred embodiment of the present invention.

FIG. 2 shows a front view of the smart protective face mask according to the preferred embodiment of the present invention of FIG. 1.

The various embodiments of the present invention will hereinafter be described in conjunction with the appended drawings.

### DETAILED DESCRIPTION

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

The following embodiments and the accompanying drawings, which are incorporated into and form part of this disclosure, illustrate embodiments of the invention and together with the description, serve to explain the principles of the invention. To the accomplishment of the foregoing and related ends, certain illustrative aspects of the invention are described herein in connection with the following description and the annexed drawings. These aspects are indicative, however, of but a few of the various ways in which the principles of the invention can be employed and the subject invention is intended to include all such aspects and their equivalents. Other advantages and novel features of the invention will become apparent from the following detailed description of the invention when considered in conjunction with the drawings.

As discussed above, embodiments of the present invention relate to a smart protective face mask that includes a power source, ventilation mechanisms, filtering mechanisms, audio and video mechanisms, headphones, a lighting mechanism, a night vision mechanism, a visual zooming mechanism, protective eyewear, a head canopy, a thermometer, a DNA testing mechanism, virus detection mechanisms, radio communication technology, and fiber optics technology.

The “smart protective face mask” was created to provide a breathable mask that amplifies the voice of the user so that he/she can adequately and clearly communicate efficiently, effectively, and accurately unlike the current masks that are available today. The “smart protective face mask” also has a built-in air ventilating system which aids in breathing for anyone who may suffer from influenza, asthma, upper respiratory breathing issues, and speech impediments as well various other talking and breathing challenges.

The provisional version of the smart protective face mask includes the following structural elements:
Head Covering that is waterproof and attachable—optional;
UV Shades Protective eyewear that has digital abilities to zoom in and out as well as night vision;
Face mask;
Inner Breathing Nostril Chambers;
Filtered Air Breathing System;
Voice box, Microphone, Amplifier;
Filtered Air Vents;
Fan System;
Head Lamp/LED Light;
An adaptable DNA Swab Tester/Attachable/Detachable—optional;
Headphones;
ADT capability; BLUETOOTH capability; connects with “SMART” cars; SMART Homes as well as any other internet base; fiber optic technologies;
Cell phones and USB capability; and
“SMART Mask” has the ability to be charged as well as run off battery energies, solar energy and WIFI.

Features include:
appropriately sized and motorized “Air Filtering Ventilation System”;
appropriately sized microphone that amplifies the voice of the consumer;
the ability to increase or decrease “Volume”;
BLUETOOTH compatible;
Sources of Power include but are not limited to rechargeable batteries, and solar panel energy;
the ability to transmit information from “smart protective face mask” to “smart protective face mask” and also, but not limited to, cell phones, computers, Smart cars and various other cyber fiber optic and internet based devices, i.e. Alexus, Google Search, Voice Command Opening, and Starting Smart cars;
Attachable/Detachable eye protective gear apparatus to deflect UV Rays ultimately to protect optical orifices;
The eye protective gear will slide up from an inner housing of the “smart protective face mask” similar to car windows sliding up and down on a vehicle “Optional” or can attach/detach;
compatibility with hearing aid/ADT system;
equipped with thermometers;
equipped with detachable/attachable “Head Canopy” that is weather proof and durable, yet breathable for the consumer;
an inner air breathing component system consisting of breathing tubing that comfortably rests partly in the consumers' nostril cavity to channel/filter fresh air in and out of the mask;
night vision including the ability to zoom in and zoom out of focus when eye gear is engaged; and
a swab adaptor that mechanically connects to an inner air filtering unit that safely and comfortably collects DNA in the event that the consumer may need to be tested for bio health reasons such as but not limited to COVID as well as any other swab testable agents that may be detrimental to consumers health and wellness.

The “Smart Mask” may also include a climate control regulator using nano-technology solar energy wave frequencies and that has the ability to adapt to the climate. For example, in the event of a blizzard the climate control regulator would modify visibility in the ocular axis as well as in “Smart mask” eye gear creating visual clarity in the blizzard conditions as well as night vision assistance if needed. The climate control regulator also would provide steamed oxygen through air filtering systems and through self-regulated temperature control. The carbon provided by the host is reconfigured through the air filtering systems maintaining the body temperature of the host which would produce a steam-like mist in their particular climate/terrain”. In a completely different climate, for example extremely hot climates, the “Smart Mask” would not overheat. The “Smart Mask” climate control regulator would keep the mask “Cool” through the air filter system's nano-technology with the carbon provided by the host which would be reconfigured and “chilled” by the micro fans system residing internally in the air filter system. Furthermore, the “Smart Mask” can be configured to be compatible with smart phones and devices similar to smart watches, and “sold” through “Apple”, “T-Mobile”, “Verizon”. Taking into account up-to-to date needs the instant mask is vital for protection against the novel Corona virus (Covid19).

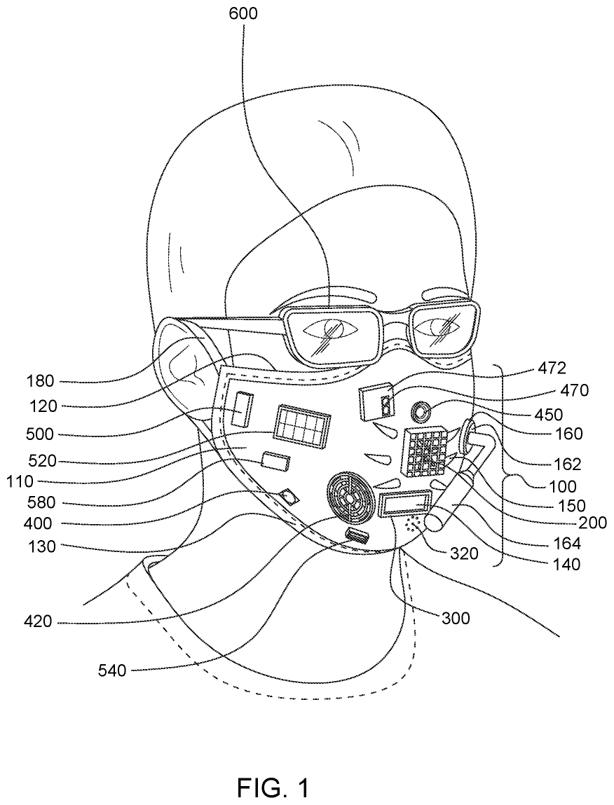

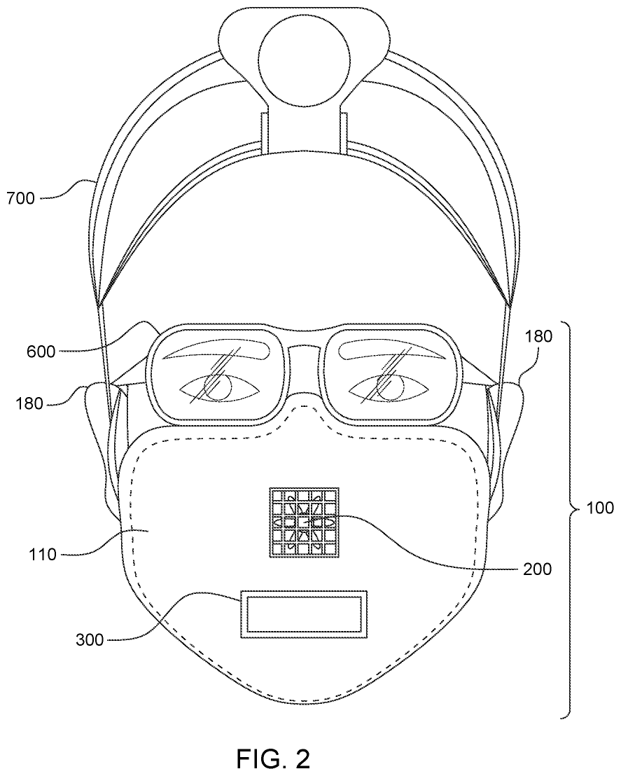

Referring now to FIGS. 1 and 2, the preferred embodiment of the face mask 100 comprises a main body 110 including a top edge portion 120 adapted to conform to the bridge of a user's nose, a bottom edge portion 130 adapted to conform to a portion of a user's chin, an oral orifice 140, a nasal orifice 150, an air supply orifice 160 adapted to releasably retain an air supply cartridge 164 therein, an air supply orifice cap 162 releasably connected to the air supply orifice and is adapted to hermetically seal the air supply orifice until the air supply orifice cap is removed, wherein the main body is adapted be placed upon a user's face and surround their nose and mouth forming an air-tight seal therebetween; at least one attachment member 180 connected to the main body and is adapted to releasably attach to a portion of the user's head to thereby releasably secure the face mask to the user's face; a ventilation mechanism 200 including an air filtering mechanism, wherein the ventilation mechanism 200 is attached and hermetically sealed to the main body 110 via the nasal orifice 150 and is adapted to filter air entering the face mask for the user to breath, and allow air exhaled by the user to exit therefrom, and may include a climate control regulator adapted to control the climate between the face mask and the face of the user; an oral mechanism 300 attached and hermetically sealed to the main body via the oral orifice, wherein the oral mechanism is adapted to digitize, amplify, and project a user's voice, wherein the oral mechanism is adapted to measure the temperature of a user's breath, and wherein the oral mechanism is adapted to detect viruses within the user's saliva and breath; protective eyewear 600 releasably attached to the top edge portion 120 of said main body 110 and is adapted to cover and protect said user's eyes when in use; a transceiver 400 attached to the main body 110, is electronically connected to the ventilation mechanism 200, is electronically connected to the oral mechanism 300, and is adapted to send and receive electronic signals to and from transceivers of external electronic devices, such as a smart phone or main frame server; and a power supply 500 attached to the main body, is electronically connected to and provides electric power to the ventilation mechanism 200, the oral mechanism 300, and the transceiver 400.

The face mask may further include a head covering 700 releasably attached to the main body 110 and be adapted to cover and protect a substantial portion of a user's head; the air supply cartridge 164 is adapted to releasably attach to and be hermetically sealed with the air supply orifice 160, such that compressed air can be delivered to the user of the facemask through the air supply orifice 160; a video mechanism 450 is attached to the main body and is electronically connected to the power supply and the transceiver; a lighting mechanism 470 attached to the main body and be electronically connected to the power supply; and a night vision mechanism 472 attached to the main body and electronically connected to said power supply.

The oral mechanism 300 may further be adapted to retrieve and analyze said user's DNA. Cotton swabs may also be incorporated therein to achieve this function. The transceiver 400 may incorporate radio technology, including BLUETOOTH. The electronic connection between the oral mechanism and the transceiver may incorporate fiber optics technology. Furthermore, the oral mechanism 300 may include a vaping cartridge attachment adapted to allow the releasable attachment and use of a vaping cartridge.

The face mask may further include climate control mechanisms, such as a cooling fan 420 attached to the main body and is adapted to circulate air between the main body and a user's face; at least one speaker 320 attached to the main body 110, and electronically connected to the power supply and the transceiver; at least one solar panel 520 attached to the main body, and electronically connected to the power supply; and at least one USB port 540 attached to the main body, and electronically connected to the power supply, the at least one speaker, and the transceiver.

The face mask may also comprising a computing mechanism 580, which can include a series of computer chips and software applications thereon, attached to the main body, and be electronically connected to the power supply, the transceiver, the ventilation mechanism, and the oral mechanism, wherein the computing mechanism is adapted to control the face mask and send and receive information to and from transceivers of external electronic devices. As such, the face mask can be controlled by the user via a smart device or a remotely controlled device.

The face mask may be formed from a flexible material, including cotton and nylon. However, the face mask may also be formed from a rigid material, such as plastic, or a semi-rigid material, such as rubber or Styrofoam, depending on the application thereof.

The face mask may also include a detachable cover over a front portion thereof that would house the features so that they will not be exposed, and act to stabilize and protect all the other components residing on and in the mask. The detachable cover can be adapted to fit on top of the frontal mouth/face mask disguising the other features. The detachable cover could be made of a light weather proof material that could also be used as carrying kit for the features of mask.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention.

Although the invention has been explained in relation to its preferred embodiment, it is to be understood that many other possible modifications and variations can be made without departing from the spirit and scope of the invention.

What is claimed is:

1. A face mask comprising:
a main body including:
a top edge portion;
wherein said top edge portion is adapted to conform to the bridge of a user's nose;
a bottom edge portion;
wherein said bottom edge portion is adapted to conform to a portion of a user's chin;
an oral orifice;
a nasal orifice;
an air supply orifice;
wherein said air supply orifice is adapted to releasably retain an air supply cartridge therein;
an air supply orifice cap;
wherein said air supply orifice cap is releasably connected to said air supply orifice and adapted to hermetically seal said air supply orifice until said air supply orifice cap is removed;
wherein said main body is adapted be placed upon a user's face and surround the nose and mouth forming an air-tight seal therebetween;
at least one attachment member;
wherein said at least one attachment member is connected to said main body and is adapted to releasably attach to a portion of said user's head to thereby releasably secure said face mask to said user's face;
a ventilation mechanism including:
an air filtering mechanism;
wherein said ventilation mechanism is attached and hermetically sealed to said main body via said nasal orifice and is adapted to filter air entering said face mask for said user to breath, and allow air exhaled by said user to exit therefrom;
an oral mechanism;
wherein said oral mechanism is attached and hermetically sealed to said main body via said oral orifice;
wherein said oral mechanism is adapted to digitize, amplify, and project a user's voice;
wherein said oral mechanism is adapted to measure the temperature of a user's breath; and
wherein said oral mechanism is adapted to detect viruses within the user's saliva and breath;
protective eyewear;
wherein said protective eyewear is releasably attached to said top edge portion of said main body and is adapted to cover and protect said user's eyes when in use;
a transceiver;
wherein said transceiver is attached to said main body, is electronically connected to said ventilation mechanism, is electronically connected to said oral mechanism, and is adapted to send and receive electronic signals to and from transceivers of external electronic devices; and
a power supply;
wherein said power supply is attached to said main body, is electronically connected to and provides electric power to said ventilation mechanism, said oral mechanism, and said transceiver.

2. The face mask of claim 1, further including a head covering releasably attached to said main body and adapted to cover and protect a substantial portion of a user's head.

3. The face mask of claim 1, further including an air supply cartridge adapted to releasably attach to and be hermetically sealed with said air supply orifice, such that compressed air can be delivered to said user of said facemask through said air supply orifice.

4. The face mask of claim 1, further including a video mechanism attached to said main body and is electronically connected to said power supply and said transceiver.

5. The face mask of claim 1, further including a lighting mechanism attached to said main body and is electronically connected to said power supply.

6. The face mask of claim 5, further including a night vision mechanism attached to said main body and is electronically connected to said power supply.

7. The face mask of claim 1, wherein said oral mechanism is further adapted to retrieve and analyze said user's DNA.

8. The face mask of claim 1, wherein said transceiver incorporates radio technology.

9. The face mask of claim 8, wherein said transceiver incorporates BLUETOOTH technology.

10. The face mask of claim 8, wherein said electronic connection between said oral mechanism and said transceiver is formed using fiber optics technology.

11. The face mask of claim 1, further including a cooling fan attached to said main body and is adapted to circulate air between said main body and a user's face.

12. The face mask of claim 1, further including at least one speaker attached to said main body, and electronically connected to said power supply and said transceiver.

13. The face mask of claim 1, further including at least one solar panel attached to said main body, and electronically connected to said power supply.

14. The face mask of claim 12, further including at least one USB port attached to said main body, and electronically connected to said power supply, said at least one speaker, and said transceiver.

15. The face mask of claim 8, wherein said oral mechanism includes a vaping cartridge attachment adapted to allow the releasable attachment and use of a vaping cartridge; and wherein said face mask includes a vaping cartridge.

16. The face mask of claim 1, further comprising a computing mechanism attached to said main body, and is electronically connected to said power supply, said transceiver, said ventilation mechanism, and said oral mechanism; and wherein said computing mechanism is adapted to control said face mask and send and receive information to and from transceivers of external electronic devices.

17. The face mask of claim 1, wherein said main body is formed from a flexible material.

18. The face mask of claim 17, wherein said flexible material is formed from a group of flexible materials consisting of cotton and nylon.

19. The face mask of claim 1, wherein said ventilation mechanism further includes a climate control regulator adapted to control the climate between said face mask and the face of said user.
